(19) United States
(12) Patent Application Publication
(10) Pub. No.: US 2025/0032708 A1
(43) Pub. Date: Jan. 30, 2025

(54) PRESSURE PROFILE BASED METRIC FOR MONITORING OPERATING STATUS OF AN INFUSION PUMP

(71) Applicant: CareFusion 303, Inc., San Diego, CA (US)

(72) Inventors: Ramkumar Subramanian, San Diego, CA (US); Daniel Abal, San Diego, CA (US); Julio Pimentel, San Diego, CA (US); Brendan Burgess, San Diego, CA (US)

(21) Appl. No.: 18/717,333

(22) PCT Filed: Dec. 9, 2021
(86) PCT No.: PCT/US2021/062574

§ 371 Date: Jun. 6, 2024

(51) Int. Cl. A61M 5/168; G16H 20/17; G16H 40/40; G16H 40/67

(52) U.S. Cl. CPC A61M 5/16854; A61M 5/16813; A61M 5/16877; G16H 20/17; G16H 40/40; G16H 40/67; A61M 2005/16868; A61M 2005/16872

(57) ABSTRACT

An infusion pump may include one or more pressure sensors. A controller associated with the infusion pump may determine, based on pressure signals from the pressure sensors, a current pressure profile for the infusion pump. The controller may further determined a first metric representative of the current pressure profile of the infusion pump. The controller may detect, based at least on a difference between the first metric and a second metric representative of a reference pressure profile of the infusion pump, one or more anomalous conditions of the infusion pump such as a valve failure. The controller may perform a corrective action in response to detecting the anomalous conditions. For example, the controller may prevent the infusion pump from performing an infusion and/or generate a notification identifying the anomalous conditions. Related systems, methods, and computer program products are also provided.

### TECHNICAL FIELD

The subject matter described herein relates generally to infusion pumps and more specifically to a pressure profile based metric for monitoring the operating status of an infusion pump.

### BACKGROUND

A fluid pump, such as an infusion pump, may be used to administer therapy to a patient by delivering nutrients, medications, blood products, or other substance to the patient. Many types of clinical treatments, such as pain management, blood glucose regulation, and chemotherapy, may require the delivery of precise volumes of fluids to a patient's circulatory system or epidural space via, for example, intravenous infusion, subcutaneous infusion, arterial infusion, epidural infusion, and/or the like. A peristaltic pump (or roller pump) is one example of a fluid pump capable of delivering precise volumes of fluids. For example, a peristaltic pump may be configured to deliver, continuously or intermittently, precisely measured doses of a fluid from a reservoir. The pumping mechanism in the peristaltic pump may include a combination of pumping fingers and occluding fingers that operate in tandem to apply pressure to sequential locations in a tubing (or other conduit) in fluid communication with the reservoir in order to drive the fluid from the reservoir to a patient.

### SUMMARY

Systems, methods, computer program products, and apparatuses are provided for monitoring the operating status of an infusion pump. In some example embodiments, there is provided a system that includes at least one processor and at least one memory. The at least one memory may include program code that provides operations when executed by the at least one processor. The operations may include: determining, based at least on one or more pressure signals from one or more pressure sensors at an infusion pump, a current pressure profile associated with the infusion pump; determining a first metric representative of the current pressure profile of the infusion pump; detecting, based at least on a difference between the first metric and a second metric representative of a reference pressure profile, one or more anomalous conditions of the infusion pump; and performing a corrective action in response to detecting the one or more anomalous conditions of the infusion pump.

In some variations, one or more features disclosed herein including the following features can optionally be included in any feasible combination. The generating of the first metric representative of the current pressure profile of the infusion pump may include applying one or more signal processing techniques.

In some variations, the one or more signal processing techniques may include a matched filter or correlator, a time domain analysis, a frequency domain analysis, and/or a time-frequency wavelet analysis.

In some variations, the one or more signal processing techniques may include a Laplace transform, a Z-transform, and a state space model (SSM).

In some variations, the one or more pressure sensors may include an upstream pressure sensor and a downstream pressure sensor. The current pressure profile of the infusion pump may be determined based at least on a first pressure signal from the upstream pressure sensor and a second pressure signal from the downstream pressure sensor.

In some variations, the first pressure signal and/or the second pressure signal comprising the current pressure profile of the infusion pump may be filtered, denoised, and/or transformed.

In some variations, the one or more anomalous conditions may include an upper occluding finger and/or a lower occluding finger of a pumping mechanism of the infusion pump failing to fully occlude a pump segment of an intravenous (IV) set loaded in the infusion pump.

In some variations, the operations may further include: adjusting, for a first time period, a flow rate of the infusion pump from a programmed flow rate to a first flow rate; and determining, based at least on a first pressure profile exhibited by the infusion pump during the first time period, the first metric.

In some variations, the operations may further include: adjusting, for a second time period, the flow rate of the infusion pump from the first flow rate to a second flow rate; and determining, based at least on a second pressure profile exhibited by the infusion pump during the second time period, the first metric.

In some variations, the operations may further include: adjusting, for a third time period, the flow rate of the infusion pump from the second flow rate back to the programmed flow rate; and determining, based at least on a third pressure profile exhibited by the infusion pump during the third time period, the first metric.

In some variations, the operations may further include: identifying the reference pressure profile based at least on a model of the infusion pump, a height of the infusion pump relative to a patient, a type of intravenous (IV) set loaded in the infusion pump, a flow rate of the infusion pump, a type of fluid being dispensed by the infusion pump, and/or a presence of an anti-siphon valve (ASV) at the infusion pump.

In some variations, the corrective action may include at least one of preventing the infusion pump from performing an infusion and generating a notification identifying the one or more anomalous conditions.

In some variations, the reference pressure profile may correspond to a pressure profile exhibited by the infusion pump while the infusion pump is operating without faults.

In some variations, the reference pressure profile may correspond to a pressure profile exhibited by the infusion pump while the infusion pump is used at a different time.

In some variations, the reference pressure profile may correspond to a pressure profile exhibited by the infusion pump while the infusion pump is used with a different patient.

In another aspect, there is provided a method for monitoring the operating status of an infusion pump. The method may include: determining, based at least on one or more pressure signals from one or more pressure sensors at an infusion pump, a current pressure profile associated with the infusion pump; determining a first metric representative of the current pressure profile of the infusion pump; detecting, based at least on a difference between the first metric and a second metric representative of a reference pressure profile, one or more anomalous conditions of the infusion pump; and performing a corrective action in response to detecting the one or more anomalous conditions of the infusion pump.

In some variations, one or more features disclosed herein including the following features can optionally be included in any feasible combination. The generating of the first metric representative of the current pressure profile of the infusion pump may include applying one or more signal processing techniques.

In some variations, the one or more signal processing techniques may include a matched filter or correlator, a time domain analysis, a frequency domain analysis, and/or a time-frequency wavelet analysis.

In some variations, the one or more signal processing techniques may include a Laplace transform, a Z-transform, and a state space model (SSM).

In some variations, the one or more pressure sensors may include an upstream pressure sensor and a downstream pressure sensor. The current pressure profile of the infusion pump may be determined based at least on a first pressure signal from the upstream pressure sensor and a second pressure signal from the downstream pressure sensor.

In some variations, the first pressure signal and/or the second pressure signal comprising the current pressure profile of the infusion pump may be filtered, denoised, and/or transformed.

In some variations, the one or more anomalous conditions may include an upper occluding finger and/or a lower occluding finger of a pumping mechanism of the infusion pump failing to fully occlude a pump segment of an intravenous (IV) set loaded in the infusion pump.

In some variations, the method may further include: adjusting, for a first time period, a flow rate of the infusion pump from a programmed flow rate to a first flow rate; and determining, based at least on a first pressure profile exhibited by the infusion pump during the first time period, the first metric.

In some variations, the method may further include: adjusting, for a second time period, the flow rate of the infusion pump from the first flow rate to a second flow rate; and determining, based at least on a second pressure profile exhibited by the infusion pump during the second time period, the first metric.

In some variations, the method may further include: adjusting, for a third time period, the flow rate of the infusion pump from the second flow rate back to the programmed flow rate; and determining, based at least on a third pressure profile exhibited by the infusion pump during the third time period, the first metric.

In some variations, the method may further include: identifying the reference pressure profile based at least on a model of the infusion pump, a height of the infusion pump relative to a patient, a type of intravenous (IV) set loaded in the infusion pump, a flow rate of the infusion pump, a type of fluid being dispensed by the infusion pump, and/or a presence of an anti-siphon valve (ASV) at the infusion pump.

In some variations, the corrective action may include at least one of preventing the infusion pump from performing an infusion and generating a notification identifying the one or more anomalous conditions.

In some variations, the reference pressure profile may correspond to a pressure profile exhibited by the infusion pump while the infusion pump is operating without faults.

In some variations, the reference pressure profile may correspond to a pressure profile exhibited by the infusion pump while the infusion pump is used at a different time.

In some variations, the reference pressure profile may correspond to a pressure profile exhibited by the infusion pump while the infusion pump is used with a different patient.

In another aspect, there is provided an infusion pump. The infusion pump may include: a pumping mechanism; one or more pressure sensors; and a controller including at least one data processor and at least one memory. The at least one memory may store instructions that causes the controllers to perform operations when executed by the at least one data processor. The operations may include: determining, based at least on one or more pressure signals from the one or more pressure sensors, a current pressure profile associated with the infusion pump; determining a first metric representative of the current pressure profile of the infusion pump; detecting, based at least on a difference between the first metric and a second metric representative of a reference pressure profile of the infusion pump, one or more anomalous conditions of the infusion pump; and performing a corrective action in response to detecting the one or more anomalous conditions of the infusion pump.

In some variations, one or more features disclosed herein including the following features can optionally be included in any feasible combination. The controller may be configured to generate the first metric representative of the current pressure profile of the infusion pump by applying one or more signal processing techniques.

In some variations, the one or more signal processing techniques may include a matched filter or correlator, a time domain analysis, a frequency domain analysis, and/or a time-frequency wavelet analysis.

In some variations, the one or more signal processing techniques may include a Laplace transform, a Z-transform, and a state space model (SSM).

In some variations, the one or more pressure sensors may include an upstream pressure sensor and a downstream pressure sensor. The current pressure profile of the infusion pump is determined based at least on a first pressure signal from the upstream pressure sensor and/or a second pressure signal from the downstream pressure sensor.

In some variations, the controller may be further configured to filter, denoise, and/or transform the first pressure signal and/or the second pressure signal comprising the current pressure profile of the infusion pump.

In some variations, the one or more anomalous conditions may include an upper occluding finger and/or a lower occluding finger of the pumping mechanism failing to fully occlude a pump segment of an intravenous (IV) set loaded in the infusion pump.

In some variations, the controller may be further configured to perform operations including: adjusting, for a first time period, a flow rate of the infusion pump from a programmed flow rate to a first flow rate; and determining, based at least on a first pressure profile exhibited by the infusion pump during the first time period, the first metric.

In some variations, the controller may be further configured to perform operations including: adjusting, for a second time period, the flow rate of the infusion pump from the first flow rate to a second flow rate; and determining, based at least on a second pressure profile exhibited by the infusion pump during the second time period, the first metric.

In some variations, the controller may be further configured to perform operations including: adjusting, for a third time period, the flow rate of the infusion pump from the second flow rate back to the programmed flow rate; and determining, based at least on a third pressure profile exhibited by the infusion pump during the third time period, the first metric.

In some variations, the controller may be further configured to identify the reference pressure profile based at least on a model of the infusion pump, a height of the infusion pump relative to a patient, a type of intravenous (IV) set loaded in the infusion pump, a flow rate of the infusion pump, a type of fluid being dispensed by the infusion pump, and/or a presence of an anti-siphon valve (ASV) at the infusion pump.

In some variations, the corrective action may include at least one of preventing the infusion pump from performing an infusion and generating a notification identifying the one or more anomalous conditions.

In some variations, the reference pressure profile may correspond to a pressure profile exhibited by the infusion pump while the infusion pump is operating without faults.

In some variations, the reference pressure profile may correspond to a pressure profile exhibited by the infusion pump while the infusion pump is used at a different time.

In some variations, the reference pressure profile may correspond to a pressure profile exhibited by the infusion pump while the infusion pump is used with a different patient.

In another aspect, there is provided a computer program product including a non-transitory computer readable medium storing instructions. The instructions may cause operations may executed by at least one data processor. The operations may include: determining, based at least on one or more pressure signals from one or more pressure sensors at an infusion pump, a current pressure profile associated with the infusion pump; determining a first metric representative of the current pressure profile of the infusion pump; detecting, based at least on a difference between the first metric and a second metric representative of a reference pressure profile, one or more anomalous conditions of the infusion pump; and performing a corrective action in response to detecting the one or more anomalous conditions of the infusion pump.

In another aspect, there is provided an apparatus. The apparatus may include: means for determining, based at least on one or more pressure signals from one or more pressure sensors at an infusion pump, a current pressure profile associated with the infusion pump; means for determining a first metric representative of the current pressure profile of the infusion pump; means for detecting, based at least on a difference between the first metric and a second metric representative of a reference pressure profile, one or more anomalous conditions of the infusion pump; and means for performing a corrective action in response to detecting the one or more anomalous conditions of the infusion pump.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a personal area network, a peer-to-peer network, a mesh network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to monitoring the operating status of an infusion pump, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of an infusion system, in accordance with some example embodiments;

FIG. 2 depicts a perspective view of an example of an infusion pump with the door of the infusion pump open, in accordance with some example embodiments;

FIG. 3A depicts an example of a finger profile illustrating the gap between the platen and the occluding fingers of an infusion pump during successive pump cycles, in accordance with some example embodiments;

FIG. 3B depicts a graph illustrating an example of a relationship between the size of the gap between platen and the occluding fingers of an infusion pump and an encoder signal from each occluding finger, in accordance with some example embodiments;

FIG. 4 depicts a graph illustrating an example of a flow rate profile, in accordance with some example embodiments;

FIG. 5 depicts an example of a matched filter and a correlator, in accordance with some example embodiments;

FIG. 6 depicts a block diagram illustrating an example of a black box representation of an infusion pump system, in accordance with some example embodiments;

FIG. 7A depicts a block diagram illustrating another example of a black box representation of an infusion pump system, in accordance with some example embodiments;

FIG. 7B depicts a schematic diagram illustrating an example of a state space model (SSM) for performing signal analysis, in accordance with some example embodiments;

FIG. 8 depicts examples of reference pressure profiles of an infusion pump operating without faults, in accordance with some example embodiments;

FIG. 9 depicts examples of pressures profiles of an infusion pump having a faulty lower occluding finger, in accordance with some example embodiments;

FIG. 10 depicts examples of pressures profiles of an infusion pump having a faulty upper occluding finger, in accordance with some example embodiments;

FIG. 11 depicts a flowchart illustrating an example of a process for monitoring the operating status of an infusion pump, in accordance with some example embodiments; and

FIG. 12 depicts a block diagram illustrating an example of a computing system, in accordance with some example embodiments.

When practical, similar reference numbers denote similar structures, features, or elements.

### DETAILED DESCRIPTION

Faults in the pumping mechanism of an infusion pump may cause the infusion pump to operate incorrectly or in an unexpected manner. For example, an infusion pump with a faulty pumping mechanism may be unable to deliver a prescribed volume of fluid and/or deliver fluid at a desired flow rate. The infusion pump may be a syringe pump or a large volume pump (LVP) such as, for example, a peristaltic pump, a piston pump, a diaphragm pump, and/or the like. In a peristaltic pump, for example, a mechanical fault that prevents the valves (e.g., occluding fingers) from fully occluding the tubing of the intravenous (IV) set loaded in the peristaltic pump may give rise to an uncontrolled flow of fluid from the peristaltic pump. Valve failures at the peristaltic pump may be attributable to a variety of causes including, for example, non-concentric pumping segments in the intravenous (IV) set, defective bezel bosses, cracked platen, broken platen assembly, and/or the like. As such, in some example embodiments, a pump controller may be configured to monitor the operating status of an infusion pump to detect a variety of anomalous conditions including those that arise from a faulty pumping mechanism.

In some example embodiments, the pump controller may monitor the operating status of the infusion pump based on a pressure profile based metric. The infusion pump may include one or more pressure sensors including, for example, an upstream pressure sensor configured to measure an upstream pressure, a downstream pressure sensor configured to measure a downstream pressure, and/or the like. The pump controller may determine, based at least on measurements from the one or more pressure sensors, a pressure profile for the infusion pump. The pressure profile for the infusion pump may include one or more static pressure signatures and/or dynamic pressure signatures associated with the presence of one or more anomalous conditions of the infusion pump. As such, the pump controller may determine, based at least on a difference between the pressure profile of the infusion pump and a reference pressure profile of the infusion pump operating without faults, a first metric indicative of the operating status of the infusion pump. For example, the first metric may correspond to a difference between a second metric whose value is representative of the pressure profile of the infusion pump and a third metric whose value is representative of the reference pressure profile. Accordingly, the pump controller may detect, based at least on whether the first metric satisfies one or more thresholds, a presence (or an absence) of anomalous conditions of the infusion pump including, for example, anomalous conditions associated with a faulty pumping mechanism such as valve failures.

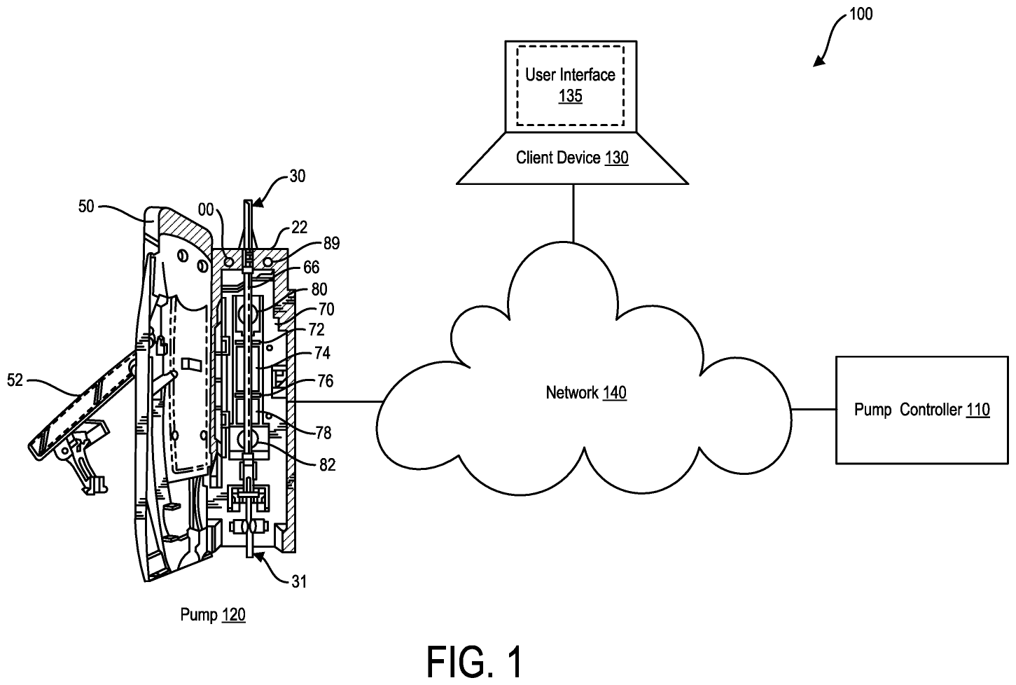

FIG. 1 depicts a system diagram illustrating an example of an infusion system 100, in accordance with some example embodiments. Referring to FIG. 1, the infusion system 100 may include a pump controller 110, a pump 120, and a client device 130. As shown in FIG. 1, the pump controller 110, the pump 120 and the client device 130 may be communicatively coupled via a network 140. The client device 130 may be a processor-based device including, for example, a point of care unit (PCU), a smartphone, a tablet computer, a wearable apparatus, a desktop computer, a laptop computer, a workstation, and/or the like. Meanwhile, the network 140 may be a wired and/or wireless network including, for example, a public land mobile network (PLMN), a local area network (LAN), a virtual local area network (VLAN), a wide area network (WAN), the Internet, and/or the like.

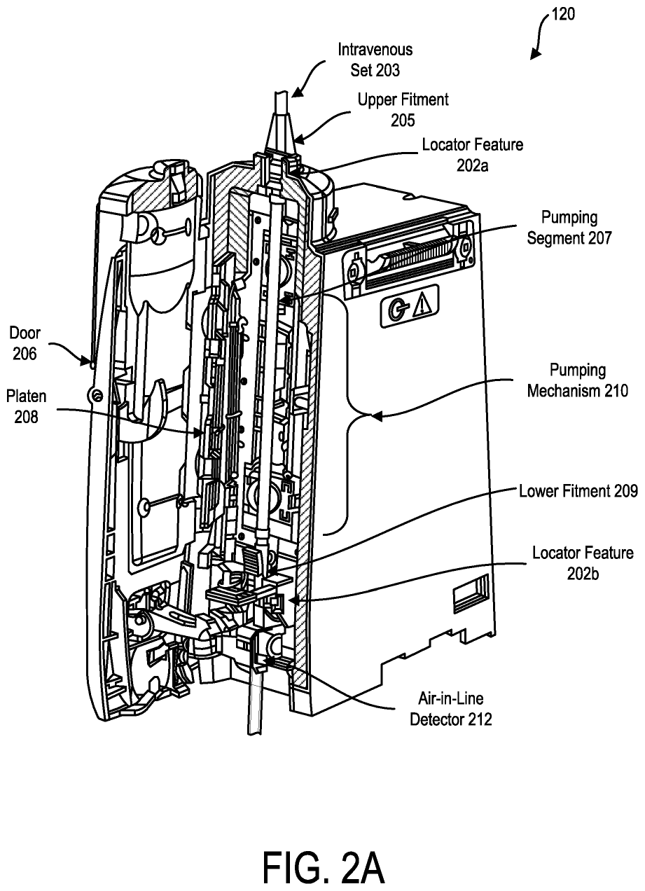

FIG. 2 depicts a perspective view of an example of the pump 120 in which a door 206 and a platen 208 of the pump 120 are in an open position. As shown in FIG. 2, the pump 120 may be loaded with an intravenous (IV) set 203 having an upper fitment 205, a pump segment 207, and a lower fitment 209. When the intravenous set 203 is loaded properly into the pump 120, the upper fitment 205 may be secured within a first locator feature 204a (e.g., a pocket) in the bezel of the pump 120 while the lower fitment 209 may be secured within a second locator feature 204b (e.g., a pocket) in the bezel of the pump 120. Moreover, when the intravenous set 203 is loaded properly into the pump 120, a portion of the tubing of the intravenous set 203 may be secured within an air-in-line (AIL) detector 212 configured to detect the presence of air within the intravenous set 203.

The pump mechanism 210 of the pump 120 may act directly on the pump segment 207 of the intravenous set 203, which connects an upstream fluid line to a downstream fluid line to form a continuous fluid conduit between a fluid reservoir and a patient. For example, the pump 120 may include a pumping mechanism 210, which may act as a flow control device moving fluid through the conduit downstream to the patient. The pump segment 207, the upstream fluid line, and/or the downstream fluid line may be coupled to a pump cassette or cartridge that is configured to be coupled to the pump 120.

Although the type of the pumping mechanism 210 may vary, the example of the pumping mechanism 210 shown in FIG. 2 is a multi-finger pumping mechanism that includes an upstream occluding finger, a primary pumping finger, a downstream occluding finger, and a secondary pumping finger. The pumping mechanism 210 may therefore operate by the pumping fingers and the occluding fingers alternately applying pressure on the pump segment 207 of the fluid conduit. The pressure may be applied at sequential locations in the pump segment 207, beginning at the upstream end of the pumping mechanism 210 and continuing through the downstream end of the pumping mechanism 210. At any one point in time, at least one of the fingers of the pumping mechanism 210 may apply a pressure that is sufficient to occlude the fluid conduit. Moreover, at least one of the fingers of the pumping mechanism 210 may not retract from occluding the pump segment 207 until a subsequent finger in the sequence has already occluded the pump segment 207. Accordingly, at no time during the operation of the pump mechanism 210 is there a direct fluid path from the reservoir to the patient.

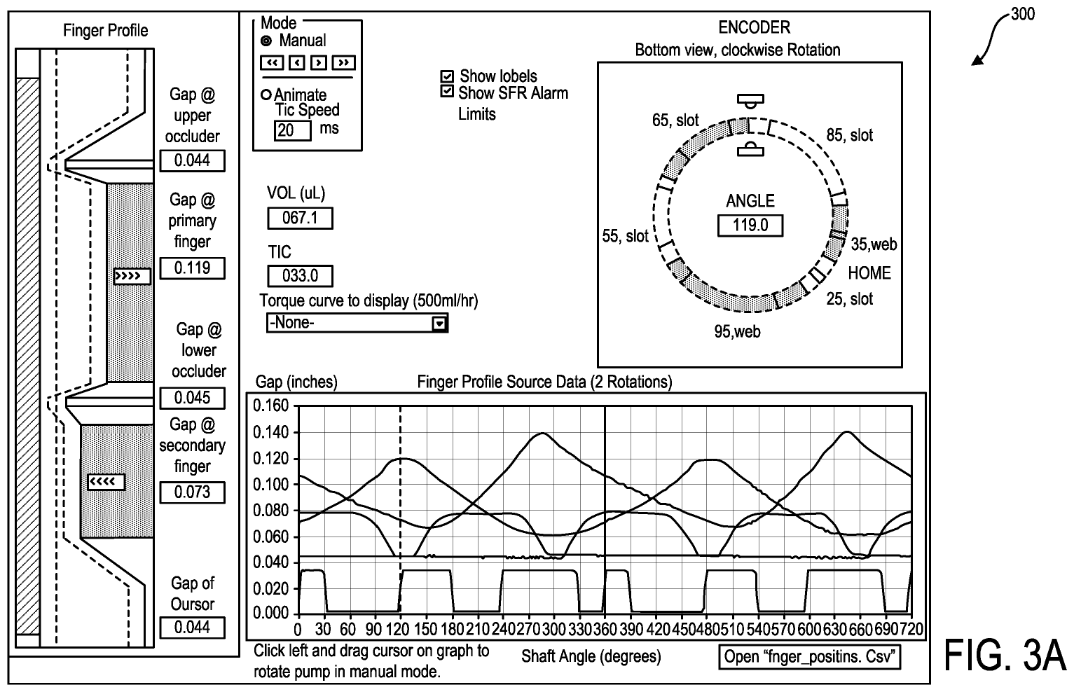

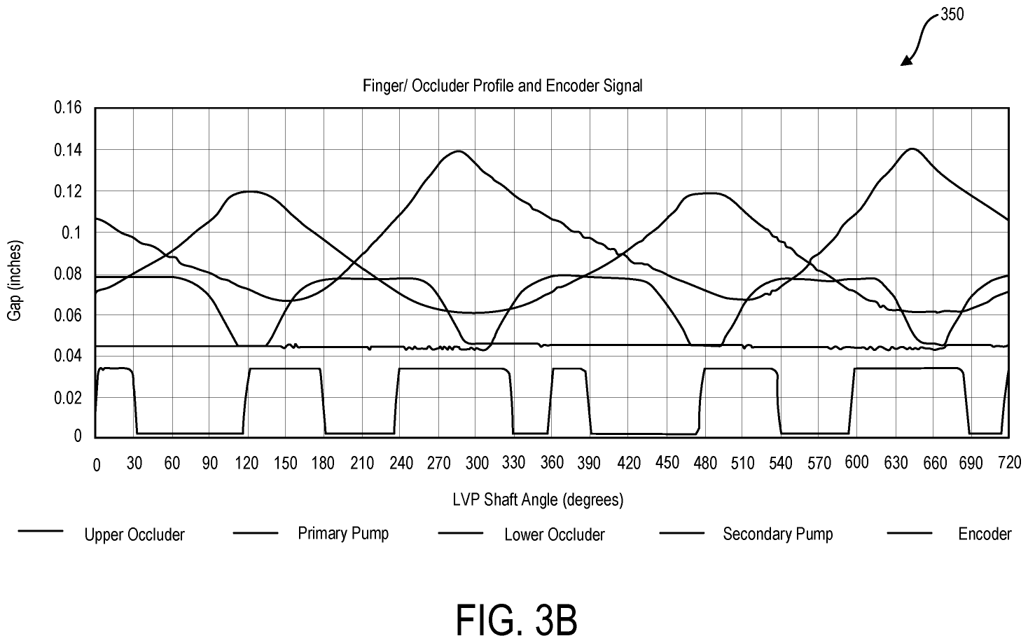

To further illustrate, FIG. 3A depicts an example of a finger profile 300 associated with the pump mechanism 210 of the pump 120. As shown in FIG. 3A, the gap between the platen 208 on one side and each occluding finger of the pump mechanism 210 on the other side may vary as the pump mechanism 120 rotates through each full pump cycle. The changes in the size of the gaps between the platen 208 and the occluding fingers of the pump mechanism 210 over two rotations of the pump mechanism 120 (e.g., 720 degrees) and the corresponding encoder signals for the upper occluding finger, the primary pump, the lower occluding finger, and the secondary pump are shown in the graph 350 depicted in FIG. 3B. As shown in FIG. 3, the pump mechanism 210 may be configured such that each occluding finger at its lowest gap position fully occludes the pump segment 207 of the intravenous set 203. In some instances, the pump 120 may be configured to operate in a burst mode in which the pump 120 delivers fluid at or below a threshold flow rate (e.g., ≤40 milliliters per hour) and/or in a continuous mode in which the pump 120 delivers fluid above a threshold flow rate (e.g., >40 milliliters per hour)

In some example embodiments, the pump controller 110 may monitor the operating status of the infusion pump 120 to detect when one or more anomalous conditions are present at the infusion pump 120. Some anomalous conditions may arise from malfunction of the pumping mechanism 210 such as, for example, a valve failure in which the pumping mechanism 210 fails to fully occlude the pump segment 207. Anomalous conditions such as valve failures may cause an uncontrolled flow of fluid from the infusion pump 120 such that the pump 120 is unable to deliver a prescribed volume of fluid at a desired flow rate and/or deliver fluid at a desired flow rate. Accordingly, the pump controller 110 may perform one or more corrective actions in response to detecting an anomalous condition of the pump 120. For example, the pump controller 110 may prevent the pump 120 from performing an infusion when an anomalous condition is detected at the pump 120. Alternatively and/or additionally, the pump controller 110 may generate a notification (e.g., an alert, an error message, and/or the like) indicating the presence of the anomalous condition of the pump 120. In some cases, the notification may identify the type and/or location of the anomalous condition of the pump 120 as well as actions for resolving the anomalous condition.

In some example embodiments, the pump controller 110 may monitor the operating status of the pump 120 based on a pressure profile based metric. The pump 120 may include one or more pressure sensors including, for example, an upstream pressure sensor configured to measure an upstream pressure, a downstream pressure sensor configured to measure a downstream pressure, and/or the like. The pump controller 110 may determine, based at least on measurements from the one or more pressure sensors, a pressure profile for the pump 120. The pressure profile for the pump 120 may include one or more static pressure signatures and/or dynamic pressure signatures indicative of the presence of one or more anomalous conditions of the pump 120. As such, the pump controller 110 may determine, based at least on a difference between the pressure profile of the pump 120 and a reference pressure profile, a first metric indicative of the operating status of the pump 120.

In some example embodiments, the first metric may correspond to a difference between a second metric whose value is representative of the pressure profile of the pump 120 and a third metric whose value is representative of the reference pressure profile. In some cases, the reference pressure profile may be the pressure profile exhibited by the pump 120 when the pump 120 is operating without faults. Alternatively and/or additionally, the reference pressure profile may be the pressure profile exhibited by the pump 120 when the pump 120 was used at a different (e.g., earlier) time and/or with a different patient. Accordingly, the pump controller 110 may detect, based at least on whether the first metric satisfies one or more thresholds, a presence (or an absence) of anomalous conditions of the pump 120 including, for example, anomalous conditions associated with faults at the pumping mechanism 210. For instance, the pump controller 110 may detect an anomalous condition of the pump 120 if the difference between the second metric whose value is representative of the pressure profile of the pump 120 and the third metric whose value is representative of the reference pressure profile exceeds a threshold.

It should be appreciated that absent any faults at the pump 120, the pressure profile of the pump 120 may nevertheless be affected by a variety of factors including, for example, flow rate, type of fluid being dispensed (e.g., fluids having different viscosities), the presence of certain mechanical features (e.g., anti-siphon valve (ASV)), and/or the like. For example, the pump 120 may exhibit a first pressure profile when operating at a first flow rate to dispense a first type of fluid and a second pressure profile when operating at a second flow rate to dispense a second type of fluid. The pump 120 may also exhibit different pressure profiles when operating with an anti-siphon valve (ASV) than when operating without an anti-siphon valve. As such, the pump 120 may be associated with multiple reference pressure profiles, each of which corresponding to a different combination of factors that may affect the pressure profile of the pump 120 absent any faults. Moreover, to analyze the pressure profile of the pump 120, the pump controller 110 may first identify a suitable reference pressure profile based on one or more of the factors present at the pump 120 (e.g., flow rate, type of fluid being dispensed, mechanical features of the pump, model of the pump, fluid administration set used to deliver fluid via the pump, height of the pump relative to the patient, or the like).

In some example embodiments, the pump controller 110 may monitor, based on the pressure profile based metric of the pump 120, the operating status of the pump 120 on a continuous basis. For example, as noted, the pressure profile based metric may correspond to a difference between one metric whose value is representative of the pressure profile of the pump 120 and another metric whose value is representative of a reference pressure profile. The pressure profile based metric may enable a rapid and efficient comparison between a current state of the pump 120 and a reference state such as when the pump 120 is operating without faults. In particular, evaluating the pressure profile based metric of the pump 120 obviates a complex analysis between the pressure signals of the pump 120 in its current state and those from a reference state.

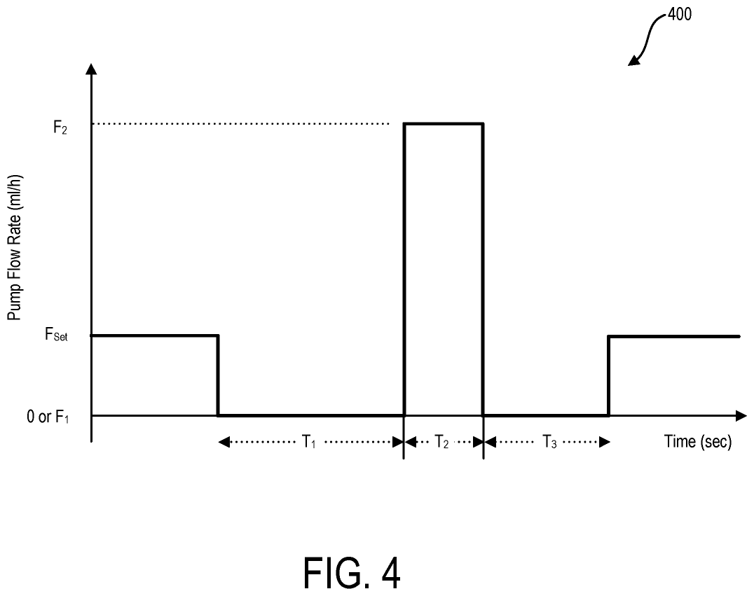

In some cases, the pump controller 110 may support a diagnostic mode in which the pump controller 110 monitors the pressure profile of the pump 120 (and the corresponding pressure profile based metric) while changing the flow rate at the pump 120 over a short period of time. To further illustrate, FIG. 4 depicts a graph illustrating an example of a flow rate profile 400 of a diagnostic mode in which the pump controller 110 changes the flow rate of the pump 120 in order to detect abnormalities in the pressure profile of the pump 120. As shown in FIG. 4, the pump controller 110 may change the flow rate of the pump 120 from a programmed flow rate Fₛₑₜ to a first flow rate F₁ for a first time period of T₁. In some instances, the first flow rate F₁ may be zero milliliters per hour (e.g., 0 ml/hour) or another flow rate that is much lower than the programmed flow rate Fₛₑₜ. In the example of the flow rate profile 400 shown in FIG. 4, the pump controller 110 in the diagnostic mode may then change the flow rate of the pump 120 from the first flow rate F₁ to a second flow rate F₂ for a second time period T₂. The second time period T₂ may be a short time period lasting between tens of milliseconds to one second. Meanwhile, the second flow rate F₂ may be a much higher flow rate than the programmed flow rate Fₛₑₜ. It should be appreciated that the magnitude of the second flow rate F₂ and the length of the second time period T₂ may be selected such that the volume of fluid infused over the second time period T₂ (e.g., ΔV=F₂×T₂) satisfy a threshold quantity (e.g., at least an x number of microliters).

Referring again to FIG. 4, the pump controller 110 in the diagnostic mode may further change the flow rate of the pump 120 from the second flow rate F₂ back to the first flow rate F₁ for a third time period T₃. In some example embodiments, the magnitudes of the first flow rate F₁ and the second flow rate F₂ and the lengths of the first time period T₁, the second time period T₂, and the third time period T₃ may be selected to satisfy Equation (1) below.

$$( {T}_{1} + {T}_{2} + {T}_{3} ) \times {F}_{set} = [ ( {T}_{1} + {T}_{3} ) \times {F}_{1} ] + ( {T}_{2} \times {F}_{2} ) \qquad ( 1 )$$

If the first flow rate F₁ is zero milliliters per hour (e.g., 0 ml/h), Equation (1) reduces to Equation (2) below.

$$( {T}_{1} + {T}_{2} + {T}_{3} ) \times {F}_{set} = {T}_{2} \times {F}_{2} \qquad ( 1 )$$

At the end of the third time period T₃, the pump controller 110 may change the flow rate of the pump 120 back to the programmed flow rate Fₛₑₜ. At that point, the pump controller 110 may exit the diagnostic mode and the pump 120 may continue to deliver fluids at the programmed flow rate Fₛₑₜ if the pump controller 110 determines that the pressure profile of the pump 120 subjected to the flow rate profile 400 does not exhibit any above-threshold deviations from a reference pressure profile of the pump 120 operating without faults when subjected to the same flow rate profile 400. For example, the pump controller 110 may determine, based at least on a difference between the pressure profile of the pump 120 and the reference pressure profile of the pump 120 operating without faults, a metric indicative of the operating status of the pump 120. Moreover, the pump controller 110 may determine, based at least on the metric, whether one or more anomalous conditions are present at the pump 120. In the event the metric indicates the presence of one or more anomalous conditions of the pump 120, such as a fault with the pumping mechanism 210, the pump controller 110 may perform one or more corrective actions. For instance, instead of allowing the pump 120 to continue delivering fluids at the programmed flow rate Fₛₑₜ, the pump controller 110 may prevent the pump 120 from performing an infusion when one or more anomalous conditions are detected at the pump 120. Alternatively and/or additionally, the pump controller 110 may generate a notification (e.g., an alert, an error message, and/or the like) indicating the presence of the one or more anomalous conditions of the pump 120.

As noted, the pump controller 110 may generate a pressure profile for the pump 120 based on measurements from the one or more pressure sensors (e.g., upstream pressure sensor, downstream pressure sensor, and/or the like) at the pump 120. However, the pressure signals from the one or more pressure sensors may exhibit low detection sensitivity when the flow rate at the pump 120 is low (e.g., at or below 1 milliliter per hour). For example, when the flow rate of the pump 120 is low, the signal-to-noise ratio (SNR) of the pressure signals from the one or more pressure sensors may be very small at low to medium flow error rates (e.g., few hundredth percent or less). As such, the pressure profile of the pump 120 at low flow rates (e.g., 1 milliliter per hour) and low flow error rates may not be easily discernable.

In some example embodiments, the pump controller 110 may apply, to a pressure signal from the one or more pressure sensors at the pump 120, one or more signal processing techniques configured to improve the detection sensitivity of the pressure signal. Doing so may enable a subsequent comparative analysis between the pressure profile of the pump 120 and a reference pressure profile of the pump 120 operating without faults and the generation of the corresponding pressure profile based metrics. For example, the pump controller 110 may apply, to the pressure signal from the one or more pressure sensors at the pump 120, one or more signal-based signal processing techniques such as matched filter (or correlator), time domain analysis, frequency domain analysis, time-frequency wavelet analysis, and/or the like. Alternatively and/or additionally, the pump controller 110 may apply, to the pressure signal from the one or more pressure sensors at the pump 120, one or more systems-based signal processing techniques such as a Laplace transform, a Z-transform (e.g., filter coefficients), a state space model, and/or the like.

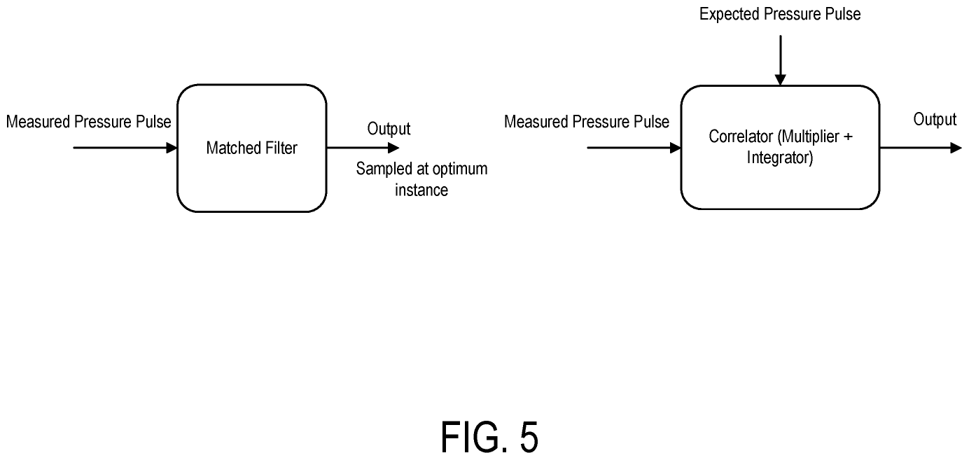

When applying the one or more signal-based signal processing techniques, the pressure signal received from the one or more pressure sensors at the pump 120 may be filtered, denoised, and/or transformed before the pump controller 110 measures and analyzes certain metrics and parameters of the resultant signal to detect one or more anomalous conditions of the pump 120. A matched filter (or correlator) is one example of a signal-based signal processing technique in which the matched filter and correlator are optimal receivers in the sense that the matched filter and correlator are configured to maximize the pulse signal-to-noise ratio (SNR) in the presence of additive white Gaussian noise. An example of a matched filter and an example of a correlator consistent with implementations of the current subject matter are depicted in FIG. 5. As shown in FIG. 5, the matched filter is indeed a filter while the correlator is a multiplier followed by an integrator. The waveforms at the output of the matched filter and correlator are usually different from one another. However, at the best sampling instant, the signal-to-noise ratio of the waveform output by the matched filter and that of the waveform output by the correlator are the same.

Another example of a signal-based signal processing technique that the pump controller 110 may apply is time domain analysis including, for example, a discrete time domain analysis in which a control system is represented as a linear difference equation of functions of time and its solution. Based on the pressure profiles of the pump 120 operating with faults and without faults, the system of interest may be modeled as a linear time-invariant with a second order under-damped response. The characteristic equation (e.g., transfer function) of such a system may correspond to Equation (3) below, which may respond to any control input signal such as a unit step, a unit impulse, and/or the like. The goal of a time domain analysis may be to measure the various time domain metrics and specification, such as delay time, peak time, rise time, setting time, peak overshoot, steady state error, natural frequency, damped frequency and damping ratio, exhibited by the pressure profiles of the pump 120 operating without faults and without faults (e.g., a valve failure and/or the like).

$$\frac{C ( s )}{R ( S )} = \frac{{w}_{n}^{2}}{s 2 + 2 δ {w}_{n} s + {w}_{n}^{2}} \qquad ( 3 )$$

Frequency domain analysis is another example of a signal-based signal processing technique that the pump controller 110 may apply to the pressure signals received from the one or more pressure sensors at the pump 120. A time domain signal may be converted into a spectral domain by applying Fourier transforms. The goal of a frequency domain analysis may be measure the various spectral domain metrics and specifications, such as resonant frequency, bandwidth, resonant peak, magnitude, and phase, exhibited by the pressure profiles of the pump 120 operating without faults and without faults (e.g., a valve failure and/or the like). A spectral estimation technique, such as periodogram and Welch's, may be applied to determine the distribution of signal power across the frequency domain.

Time-frequency analysis is yet another example of a signal-based signal processing technique that the pump controller 110 may apply to the pressure signals received from the one or more pressure sensors at the pump 120. Time-frequency analysis may include the application of techniques such as continuous wavelet transform (CWT) and Short-Time Fourier Transform (STFT), which may be more robust than traditional Fourier transforms when it comes to representing functions that have fast localized variations with sharp peaks and valleys, and for accurately deconstructing and reconstructing finite, non-periodic and/or non-stationary signals. Wavelet transform may offer additional has advantages over Short-Time Fourier Transform (STFT) because wavelet transform may be able to provide a better signal representation due to its multiresolution analysis capability. A wavelet may be a wave-like oscillation with an amplitude that increases from zero before decreasing back to zero. Wavelet analysis may expand an input signal in terms of wavelets but not in terms of sinusoid functions as in the case of a Fourier analysis. The pressure signals from the one or more pressure sensors at the pump 120 may exhibit localized sharp variations and are non-stationary in nature. With a time-frequency analysis, such as wavelet analysis and Short-Time Fourier Transform (STFT), the pressure signals from the one or more pressure sensors at the pump 120 may be represented as time-variant spectral power in a joint time-frequency domain.

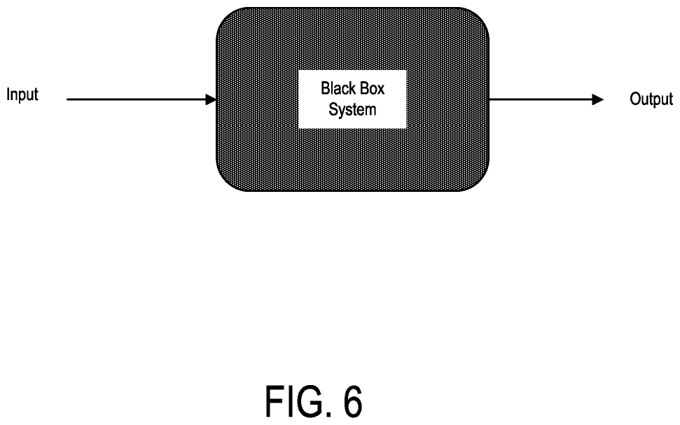

Instead of or in addition to a signal-based signal processing technique, the pump controller 110 may also apply one or more systems-based signal processing techniques. With a systems-based signal processing technique, the pump controller 110 may treat the pump 120 from which the pressure signals originate as a black box. The goal of a systems-based signal processing technique may be to identify and model the system, in this case the one or more pressures sensors at the pump 120, based on its input data and output data. To further illustrate, FIG. 6 depicts a block diagram illustrating an example of a black box representation of the pump 120. As noted, examples of systems-based signal processing methods may include a Laplace transform, a Z-transform, and a state space model (SSM).

In some example embodiments, a Z-transform is a discrete version of a Laplace transform. With a transform-based analysis, the pump controller 110 may identify and estimate the black box representation of a system, such as the pump 120, through either a first principal approach or by performing a system estimation. With a first principal approach, the pump controller 110 may hypothesize and use an electrical or mechanical model using lumped circuit elements such as resistors/capacitors or spring/mass/damper for the black box system. After identifying the system, the pump controller 110 may apply mathematical transform operators, such as a Laplace transform or a Z-transform, to identify the transfer function and determine its pole-zero locations or filter coefficients.

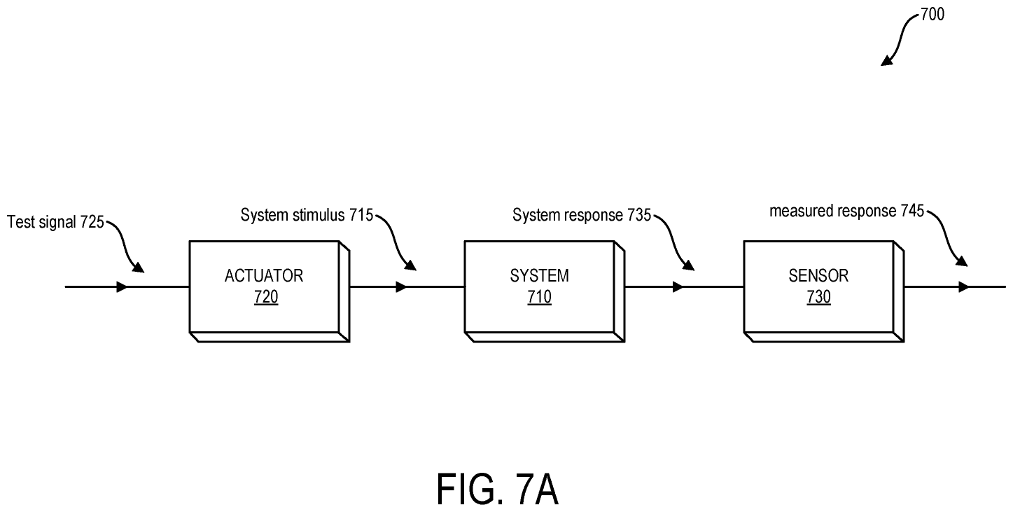

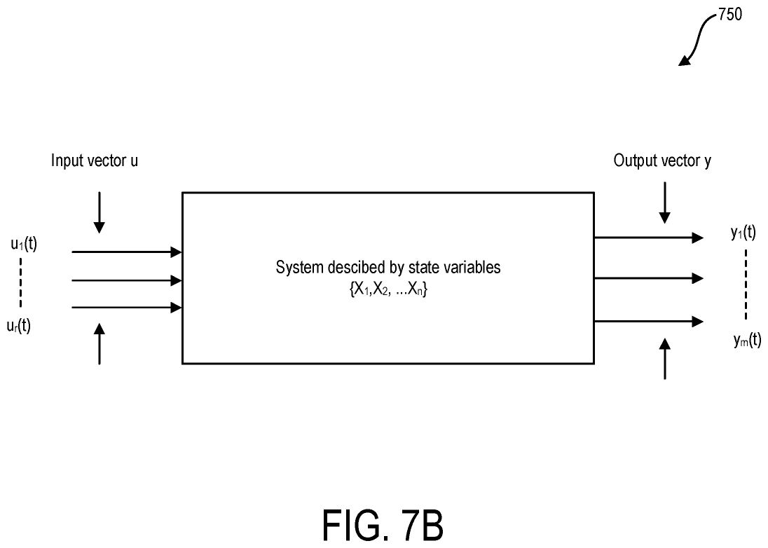

To perform a system estimation, the pump controller 110 may use one or more simulation tools to estimate the black box system using the system response and system stimulus. FIG. 7A depicts a block diagram illustrating an example of a simulation 700 in which a black box system 710 is subjected to a system stimulus 715 from an actuator 720 triggered by a test signal 725. As shown in FIG. 7A, the black box system 720 may respond to the system stimulus 715 by outputting a system response 735 that is then measured by a sensor 730 configured to provide a corresponding measured response 745. With system estimation, the pump controller 110 may observe the pressure signals from the one or more pressure sensors at the pump 120 while the pump 120 is subjected to an input stimulus that is either measured or approximated. Based on the input stimulus and the corresponding response, the pump controller 110 may fit a model for the black box system, identify its transfer function, and determine its filter coefficients or pole-zero locations.

Another example of a systems-based signal processing technique is a state space model (SSM), which represents a physical system as a set of inputs, outputs, and state variables x(t). The state space model may have an order that is equal to the dimension of state vector x(t). Moreover, the state space model may use state variables to describe a system by a set of first-order differential or difference equations instead of one or more n^{th} order differential or difference equations. State variables may be reconstructed from the measured input-output data, but are not themselves measured during an experiment. The values of state variables may evolve through time and their changes at any given time may depend on the current values of the state variables as well as the values of the input variables. Meanwhile, the values of the output variables may be contingent upon the values of the state variables and the values of the input variables.

FIG. 7B depicts a schematic diagram illustrating an example of a state space model 750 for performing signal analysis, in accordance with some example embodiments. In some example embodiments, the pump controller 110 may perform an analysis of the pressure signals output by the one or more pressure sensors at the pump 120 based on the example of the state space model 750 shown in FIG. 7B. This signal analysis may use a controls approach that allows the pump 120 to be modeled with multiple inputs and complex dynamic internal state variables accommodating head height change, backpressure, and silicone segment variation over time. A state space model, such as the state space model 750 shown in FIG. 7B, may be characterized by Equations (3) and (4) below, with Equation (3) expressing the state x of the state space model and Equation (4) expressing the observed output y of the state space model. A portion of the state space model analysis may include measuring metrics such as the transfer function matrix, eigenvalues, and eigenvectors exhibited by the pressure profiles of the pump 120 operating without faults and without faults (e.g., a valve failure and/or the like).

$$\dot{x} ( t ) = A ( t ) x ( t ) = B ( t ) u ( t ) \qquad ( 3 )$$

$$y ( t ) = C ( t ) x ( t ) + D ( t ) u ( t ) \qquad ( 4 )$$

In some example embodiments, instead of analyzing the pressure signals from the one or more pressure sensors at the pump 120 in their entirety, the pump controller 110 may perform its analysis on select portions of the pressure signals from the one or more pressure sensors at the pump 120. For example, when applying one or more detection algorithms to a pressure signal from the one or more pressure sensors at the pump 120, the pump controller 110 may concentrate on one or more regions of interest (ROI), which are portions of the pressure signal more likely to contain useful information and bear results. Moreover, prior to the application of one or more signal processing techniques, the pressure signals received from the one or more pressure sensors at the pump 120 may undergo a pre-filtering to remove high frequency noise. For instance, the pump controller 110 may apply one or more filters (e.g., a low pass filter (LPF), a bandpass filter (BPF), and/or the like) configured to preserve important features present in the pressure signal such as pressure pulses, transition shape and/or slopes (e.g., arising from valve opening or closing), and/or the like.

In some example embodiments, the pump controller 110 may apply its analysis to different regions of interest depending on the type of anomalous condition being detected at the pump 120. For example, in order to detect a fault with a lower occluding finger at the pump 120, the pump controller 110 may apply its analysis to a primary region of interest that includes a first pressure signal measured by the downstream pressure sensor during one or more fill phases of the pumping mechanism 210 at the pump 120. Although less important, the pump controller 110 may also extend its analysis to a second pressure signal measured by the upstream pressure sensor during one or more fill phases of the pumping mechanism 210 at the pump 120. The various regions of interest (ROI) for detecting faults with a lower occluding finger at the pump 120 may include a first portion of the pressure signal (from the upstream pressure sensor and/or the downstream pressure sensor) spanning a time period (approximately a few hundred milliseconds) during which each pressure pulse occurs during the fill phase, a second portion of the pressure signal spanning the entire duration of the fill phase of the pumping mechanism 210, a third portion of the pressure signal when the upper occluding finger opens, and/or a fourth portion of the pressure signal when the upper occluding finger closes.

To detect a fault with an upper occluding finger at the pump 120, the pump controller 110 may apply its analysis to a primary region of interest (ROI) that includes a first pressure signal measured by the upstream pressure sensor during one or more delivery phases of the pumping mechanism 210 at the pump 120. Although less important, the pump controller 110 may also apply its analysis to a second pressure signal measured by the downstream pressure sensor during one or more delivery phases of the pumping mechanism 210 at the pump 120. The various regions of interest (ROI) for detecting faults with an upper occluding finger at the pump 120 may include a first portion of the pressure signal (from the upstream pressure sensor and/or the downstream pressure sensor) spanning a time period (approximately a few hundred milliseconds) during which each pressure pulse occurs during the delivery phase, a second portion of the pressure signal spanning the entire duration of the delivery phase of the pumping mechanism 210, a third portion of the pressure signal when the lower occluding finger opens, and/or a fourth portion of the pressure signal when the lower occluding finger closes.

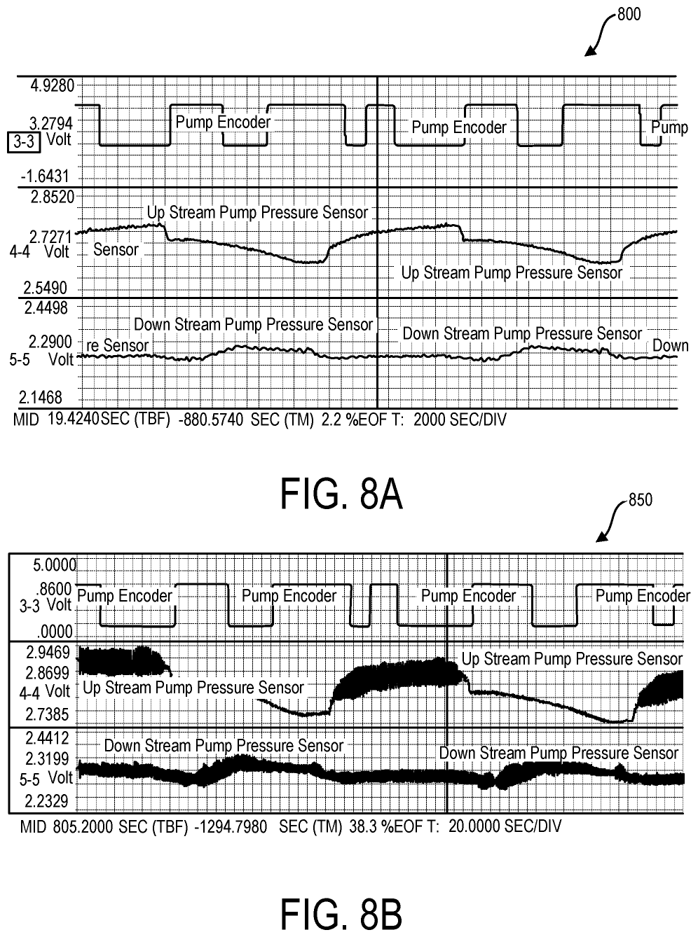

FIG. 8 depicts examples of reference pressure profiles for the pump 120 operating without faults, in accordance with some example embodiments. The examples of reference pressure profiles shown in FIG. 8 include a first pressure signal from the upstream pressure sensor at the pump 120 and a second pressure signal from the downstream pressure sensor at the pump 120. The first reference pressure profile 800 shown in FIG. 8(a) and the second reference pressure profile 850 shown in FIG. 8(b) may span over two cycles of the pumping mechanism 210. Moreover, the first reference pressure profile 800 shown in FIG. 8(a) is associated with a flow rate of 125 milliliters per hour (e.g., 125 ml/h) while the second reference pressure profile 850 shown in FIG. 8(b) is associated with a flow rate of 1 milliliter per hour (e.g., 1 ml/h). The second reference pressure profile 850 clearly depicts the pressure bursts that are associated the pump 120 operating in a burst mode at the 1 milliliter-per-hour flow rate. Nevertheless, FIG. 8 shows that the reference pressure profiles of the pump 120 may remain similar even when the pump 120 is operating at different flow rates.

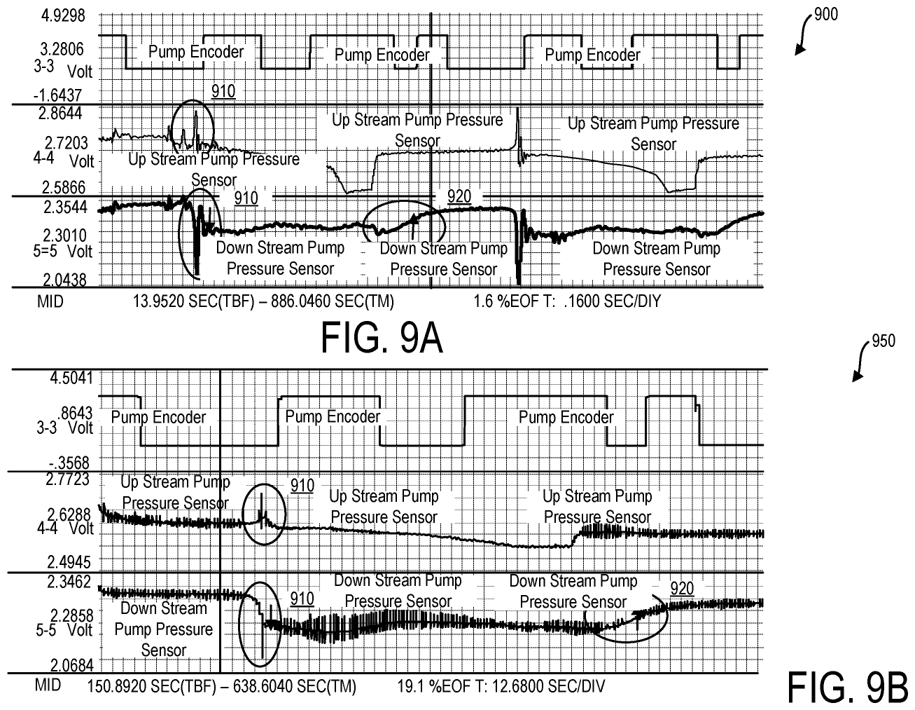

FIG. 9 depicts examples of pressures profiles of an infusion pump having a faulty lower occluding finger, in accordance with some example embodiments. The examples of pressure profiles shown in FIG. 9 include a first pressure signal from the upstream pressure sensor at the pump 120 and a second pressure signal from the downstream pressure sensor at the pump 120. Moreover, the first pressure profile 900 shown in FIG. 9(a) is associated with a flow rate of 125 milliliters per hour (e.g., 125 ml/h) while the second pressure profile 950 shown in FIG. 9(b) is associated with a flow rate of 1 milliliter per hour (e.g., 1 ml/h). In both instances, the lower occluding finger fails to fully occlude with a relatively large error (e.g., 550% at a flow rate 125 ml/h and 63000% at a flow rate of 1 ml/h).

The ellipses 910 in each pressure profile indicate portions of a pressure signal where the upper occluding finger is closing and creating a pressure signature detected by the upstream pressure sensor and the downstream pressure sensor. As shown in FIG. 9, a dynamic transitory pressure signature and a static pressure signature are observed in the pressure signal from the downstream pressure sensor. The static pressure signature, indicated by the downward arrow, shows that the measured downstream force is lower after the upper occluding finger closes than before. The ellipses 920 indicate portions of the pressure signal where the upper occluding finger is opening and creating a pressure signature detected by the downstream pressure sensor. A static pressure signature is observed herein, as indicated by the downward arrow, where the measured downstream force is higher after opening of the upper occulting finger than before. The regions marked by the ellipses 910 and 920 represent deviations from the reference pressure profile of the pump 120 operating without faults. That is, the opening and closing of the upper occluding finger when the pump 120 is operating without any faults would not create the same pressure profile at the downstream pressure sensor because the pressure pulse associated with the opening and closing of the upper occluding finger would not pass through the closed lower occluding finger.

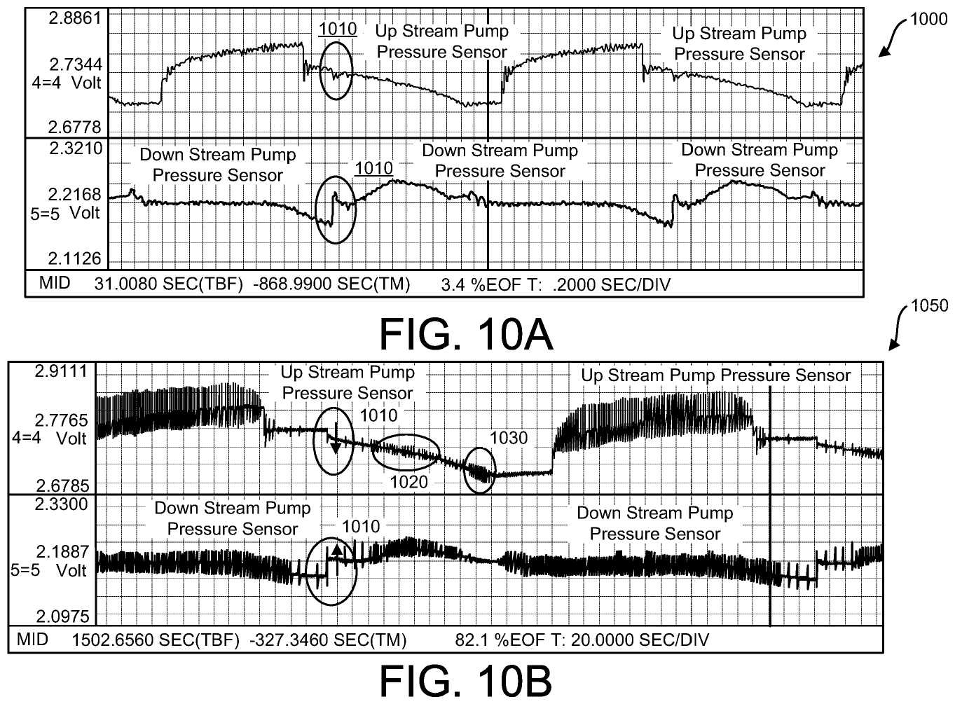

FIG. 10 depicts examples of pressures profiles of an infusion pump having a faulty upper occluding finger, in accordance with some example embodiments. The examples of pressure profiles shown in FIG. 10 include a first pressure signal from the upstream pressure sensor at the pump 120 and a second pressure signal from the downstream pressure sensor at the pump 120. Moreover, the first pressure profile 900 shown in FIG. 9(a) is associated with a flow rate of 125 milliliters per hour (e.g., 125 ml/h) while the second pressure profile 950 shown in FIG. 9(b) is associated with a flow rate of 1 milliliter per hour (e.g., 1 ml/h). In both instances, the upper occluding finger fails to fully occlude with a relatively large error (e.g., 550% at a flow rate 125 ml/h and 24500% at a flow rate of 1 ml/h).

The ellipses 1010 in FIG. 10 identifies portions of the pressure signals where the lower occluding finger is opening and creating a force signature detected by the upstream pressure sensor. The static pressure signature, indicated by the arrows, shows that the measured downstream force and upstream force undergo a sharp increase and a sharp decrease during the opening of the lower occluding finger. The ellipse 1020 identifies a portion of the pressure signal from the upstream pressure sensor that corresponds to the movement of the primary finger. The ellipse 1030 identifies a portion of the pressure signal from the upstream pressure sensor where the lower occluding finger is closing and creating a pressure signature detected by the upstream pressure sensor. The regions of the pressure signals identified by the ellipses 1010, 1020, and 1030 correspond to the delivery phase of pumping mechanism 210 during which the upper occluding finger is supposed to be fully closed and no pressure pulses related to bursts movement of the pumping mechanism should be detected by the upstream pressure sensor. As such, the regions identified by the ellipses 1010, 1020, and 1030 represent deviations from the reference pressure profile of the pump 120 operating without faults.

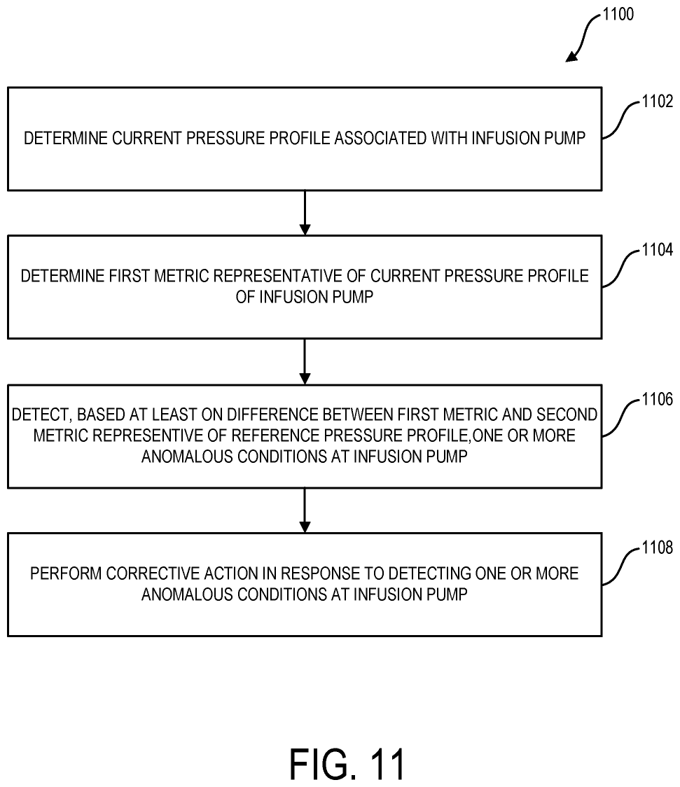

FIG. 11 depicts a flowchart illustrating an example of a process 1100 for monitoring the operating status of an infusion pump. Referring to FIGS. 1 and 11, the process 1100 may be performed by the pump controller 110 to detect and respond to a variety of anomalous conditions of the pump 120. According to some example embodiments, the pump controller 110 may determine one or more metrics representative of the pressure profiles of the pump 120 in its current state and in a reference state such as when the pump 120 is operating without faults. One or more anomalous conditions of the pump 120, including malfunctions of the pumping mechanism 210, may be detected based at least on the pressure profile based metrics.

At 1102, the pump controller 110 may determine a current pressure profile associated with an infusion pump. In some example embodiments, the pump 120 may include one or more pressure sensors including, for example, an upstream pressure sensor configured to measure an upstream pressure, a downstream pressure sensor configured to measure a downstream pressure, and/or the like. The pump controller 110 may determine, based at least on measurements from the one or more pressure sensors at the pump 120, a pressure profile for the pump 120. As shown in FIGS. 9-10, the pressure profile for the pump 120 may include a first pressure signal from the upstream pressure sensor and a second pressure signal from the downstream pressure sensor. Moreover, as shown in FIGS. 9-10, the pressure profile for the pump 120 may include pressure signals that span one or more cycles of the pumping mechanism 210 at the pump 120 to include one or more fill phases and delivery phases of the pumping mechanism 210.

At 1104, the pump controller 110 may determine a first metric representative of the current pressure profile of the infusion pump. For example, in some example embodiments, the pump controller 110 may determine a first metric whose value is representative of the pressure profile of the pump 120 in its current state. The value of the first metric may be indicative of the operating status of the pump 120. To enable the generation of this first metric and a subsequent comparative analysis to a metric whose value is representative of a reference pressure profile (e.g., of the pump 120 in a reference state), the pump controller 110 may apply one or more signal processing techniques. For instance, the pump controller 110 may apply, to the pressure signal from the one or more pressure sensors at the pump 120, one or more signal-based signal processing techniques such as matched filter (or correlator), time domain analysis, frequency domain analysis, time-frequency wavelet analysis, and/or the like. Alternatively and/or additionally, the pump controller 110 may apply, to the pressure signal from the one or more pressure sensors at the pump 120, one or more systems-based signal processing techniques such as a Laplace transform, a Z-transform (e.g., filter coefficients), a state space model, and/or the like.

conditions of At 1106, the pump controller 110 may detect, based at least on a difference between the first metric and a second metric representative of a reference pressure profile, one or more anomalous conditions of the infusion pump. As shown in FIGS. 8-10, the pressure profile of the pump 120 operating with one or more faults, such as malfunctions of the pumping mechanism 210, may exhibit distinctive pressure signatures that deviate from the reference pressure profile of the pump 120 operating without faults. Accordingly, an above-threshold difference between the first metric whose value is representative of the pressure profile of the pump 120 in its current state and a second metric whose value is representative of a reference pressure profile of the pump 120 (e.g., operating without faults), may indicate the presence (or absence) of one or more anomalous conditions of the pump 120. conditions of Examples of anomalous conditions include malfunctions of the pumping mechanism 210 such as one or more of the occluding fingers of the pumping mechanism 210 failing to fully occlude the pump segment 207 of the intravenous set 203. As noted, anomalous conditions such as valve failures may cause an uncontrolled flow of fluid from the infusion pump 120 such that the pump 120 is unable to deliver a prescribed volume of fluid at a desired flow rate and/or deliver fluid at a desired flow rate.

At 1108, the pump controller 110 may perform a corrective action in response to detecting the one or more anomalous conditions of the infusion pump. In some example embodiments, the pump controller 110 may perform one or more corrective actions when the pressure profile based metric indicates the presence one or more anomalous conditions of the pump 120. For example, the pump controller 110 may prevent the pump 120 from performing an infusion when one or more anomalous conditions are detected at the pump 120. Alternatively and/or additionally, the pump controller 110 may generate, for display at the pump 120 and/or a user interface 135 at the client device 130, a notification (e.g., an alert, an error message, and/or the like) indicating the presence of the one or more anomalous conditions of the pump 120. In some cases, the notification may identify the type and/or location of the anomalous condition of the pump 120 as well as provide instructions for resolving the anomalous conditions.

In some example embodiments, the pump controller 110 may trigger the one or more corrective actions at the pump 120 by at least transmitting one or more messages to adjust an operational state or functional element of the pump 120. The message may include specific instructions to be executed by a processor of the device to manifest the change. The corrective action may include storing a value in a location of a storage device for subsequent retrieval by the pump 120, transmitting a value directly to the pump 120 via at least one wired or wireless communication medium, transmitting or storing a reference to a value, and the like. For example, a control message may include a value to adjust a level of power from a power source of the pump 120. As another example, a control message may activate or deactivate a structural element of the pump 120 such as a light, audio playback, a motor, a lock, a pumping mechanism, a power supply, a display, or another component of the pump 120. A corrective action may include indirect control of the pump 120 by adjusting a configuration value used by the pump 120. For example, the control message may include a threshold value for a device characteristic (e.g., temperature, rate, frequency, etc.). The threshold value may be stored in a memory location and referred to by the pump 120 during operation.

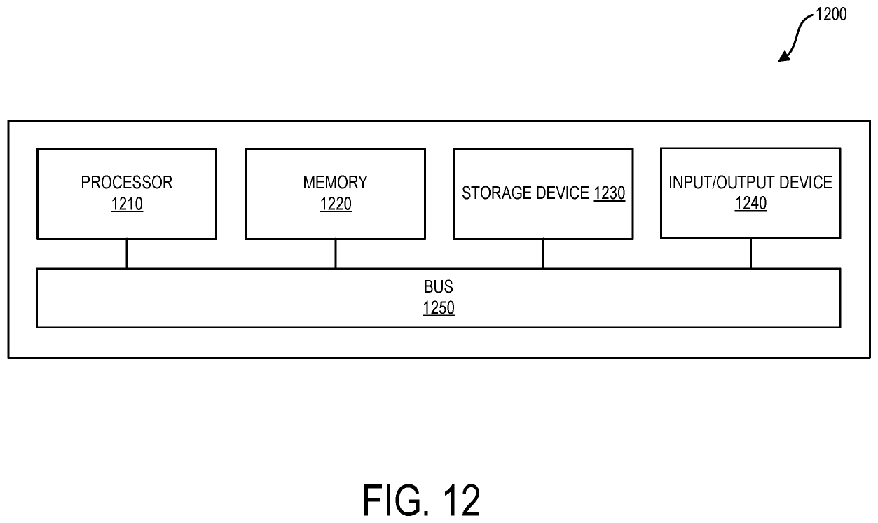

FIG. 12 depicts a block diagram illustrating a computing system 1200 consistent with implementations of the current subject matter. Referring to FIGS. 1 and 12, the computing system 1200 can be used to implement the pump controller 110 and/or any components therein.

As shown in FIG. 12, the computing system 1200 can include a processor 1210, a memory 1220, a storage device 1230, and an input/output device 12401240. The processor 1210, the memory 1220, the storage device 1230, and the input/output device 1240 can be interconnected via a system bus 1250. The processor 1210 is capable of processing instructions for execution within the computing system 1200. Such executed instructions can implement one or more components of, for example, the pump controller 110. In some example embodiments, the processor 1210 can be a single-threaded processor. Alternatively, the processor 1210 can be a multi-threaded processor. The processor 1210 is capable of processing instructions stored in the memory 1220 and/or on the storage device 1230 to display graphical information for a user interface provided via the input/output device 1240.

The memory 1220 is a computer readable medium such as volatile or non-volatile that stores information within the computing system 1200. The memory 1220 can store data structures representing configuration object databases, for example. The storage device 1230 is capable of providing persistent storage for the computing system 1200. The storage device 1230 can be a floppy disk device, a hard disk device, an optical disk device, a tape device, a solid-state device, and/or any other suitable persistent storage means. The input/output device 1240 provides input/output operations for the computing system 1200. In some example embodiments, the input/output device 1240 includes a keyboard and/or pointing device. In various implementations, the input/output device 1240 includes a display unit for displaying graphical user interfaces.

According to some example embodiments, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet).

In some example embodiments, the computing system 1200 can be used to execute various interactive computer software applications that can be used for organization, analysis and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1240. The user interface can be generated and presented to a user by the computing system 1200 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions which, when executed by the at least one data processor, result in operations comprising:
determining, based at least on one or more pressure signals from one or more pressure sensors at an infusion pump, a current pressure profile associated with the infusion pump;
determining a first metric representative of the current pressure profile of the infusion pump;
detecting, based at least on a difference between the first metric and a second metric representative of a reference pressure profile, one or more anomalous conditions of the infusion pump; and
performing a corrective action in response to detecting the one or more anomalous conditions of the infusion pump.

2. The system of claim 1, wherein the generating of the first metric representative of the current pressure profile of the infusion pump includes applying one or more signal processing techniques.

3. The system of claim 2, wherein the one or more signal processing techniques include a matched filter or correlator, a time domain analysis, a frequency domain analysis, and/or a time-frequency wavelet analysis.

4. The system of claim 2, wherein the one or more signal processing techniques include a Laplace transform, a Z-transform, and a state space model (SSM).

5. The system of claim 1, wherein the one or more pressure sensors include an upstream pressure sensor and a downstream pressure sensor, and wherein the current pressure profile of the infusion pump is determined based at least on a first pressure signal from the upstream pressure sensor and a second pressure signal from the downstream pressure sensor.

6. The system of claim 5, wherein the first pressure signal and/or the second pressure signal comprising the current pressure profile of the infusion pump are filtered, denoised, and/or transformed.

7. The system of claim 1, wherein the one or more anomalous conditions include an upper occluding finger and/or a lower occluding finger of a pumping mechanism of the infusion pump failing to fully occlude a pump segment of an intravenous (IV) set loaded in the infusion pump.

8. The system of claim 1, wherein the operations further comprise:
adjusting, for a first time period, a flow rate of the infusion pump from a programmed flow rate to a first flow rate; and
determining, based at least on a first pressure profile exhibited by the infusion pump during the first time period, the first metric.

9. The system of claim 8, wherein the operations further comprise:
adjusting, for a second time period, the flow rate of the infusion pump from the first flow rate to a second flow rate; and
determining, based at least on a second pressure profile exhibited by the infusion pump during the second time period, the first metric.

10. The system of claim 9, wherein the operations further comprise:
adjusting, for a third time period, the flow rate of the infusion pump from the second flow rate back to the programmed flow rate; and
determining, based at least on a third pressure profile exhibited by the infusion pump during the third time period, the first metric.

11. The system of claim 1, wherein the operations further comprise:
identifying the reference pressure profile based at least on a model of the infusion pump, a height of the infusion pump relative to a patient, a type of intravenous (IV) set loaded in the infusion pump, a flow rate of the infusion pump, a type of fluid being dispensed by the infusion pump, and/or a presence of an anti-siphon valve (ASV) at the infusion pump.

12. The system of claim 1, wherein the corrective action includes at least one of preventing the infusion pump from performing an infusion and generating a notification identifying the one or more anomalous conditions.

13. The system of claim 1, wherein the reference pressure profile corresponds to a pressure profile exhibited by the infusion pump while the infusion pump is operating without faults.

14. The system of claim 1, wherein the reference pressure profile corresponds to a pressure profile exhibited by the infusion pump while the infusion pump is used at a different time.

15. The system of claim 1, wherein the reference pressure profile corresponds to a pressure profile exhibited by the infusion pump while the infusion pump is used with a different patient.

16. A computer-implemented method, comprising:
determining, based at least on one or more pressure signals from one or more pressure sensors at an infusion pump, a current pressure profile associated with the infusion pump;
determining a first metric representative of the current pressure profile of the infusion pump;
detecting, based at least on a difference between the first metric and a second metric representative of a reference pressure profile, one or more anomalous conditions of the infusion pump; and
performing a corrective action in response to detecting the one or more anomalous conditions of the infusion pump.

17. (canceled)

18. (canceled)

19. (canceled)

20. (canceled)

21. (canceled)

22. The system of claim 16, wherein the one or more anomalous conditions include an upper occluding finger and/or a lower occluding finger of a pumping mechanism of the infusion pump failing to fully occlude a pump segment of an intravenous (IV) set loaded in the infusion pump.

23. The system of claim 16, further comprising:
adjusting, for a first time period, a flow rate of the infusion pump from a programmed flow rate to a first flow rate; and
determining, based at least on a first pressure profile exhibited by the infusion pump during the first time period, the first metric.

24. (canceled)

25. (canceled)

26. (canceled)

27. (canceled)

28. (canceled)

29. (canceled)

30. (canceled)

31. An infusion pump, comprising:
a pumping mechanism;
one or more pressure sensors; and
a controller comprising at least data processor and at least one memory storing instructions which, when executed by the at least one data processor, cause the controller to perform operations comprising:
determining, based at least on one or more pressure signals from the one or more pressure sensors, a current pressure profile associated with the infusion pump;
determining a first metric representative of the current pressure profile of the infusion pump;
detecting, based at least on a difference between the first metric and a second metric representative of a reference pressure profile of the infusion pump, one or more anomalous conditions of the infusion pump; and
performing a corrective action in response to detecting the one or more anomalous conditions of the infusion pump.

32. (canceled)

33. (canceled)

34. (canceled)

35. (canceled)

36. (canceled)

37. The infusion pump of claim 31, wherein the one or more anomalous conditions include an upper occluding finger and/or a lower occluding finger of the pumping mechanism failing to fully occlude a pump segment of an intravenous (IV) set loaded in the infusion pump.

38.-48. (canceled)
